(19) United States
(12) Patent Application Publication
(10) Pub. No.: US 2017/0298446 A1
(43) Pub. Date: Oct. 19, 2017

(54) BIOMARKERS OF BRUTON TYROSINE KINASE INHIBITOR RESISTANCE

(71) Applicant: OHIO STATE INNOVATION FOUNDATION, COLUMBUS, OH (US)

(72) Inventors: John BYRD, Columbus, OH (US); Amy JOHNSON, Columbus, OH (US); Gerard LOZANSKI, Dublin, OH (US); Arletta LOZANSKI, Dublin, OH (US); Jennifer WOYACH, Columbus, OH (US)

(73) Assignee: Ohio State Innovation Foundation, Columbus, OH (US)

(21) Appl. No.: 15/516,088

(22) PCT Filed: Oct. 5, 2015
(86) PCT No.: PCT/US2015/053963

§ 371 Date: Mar. 31, 2017

Related U.S. Application Data
(60) Provisional application No. 62/059,501, filed on Oct. 3, 2014.

(51) Int. Cl. C12Q 1/68; A61K 31/519

(52) U.S. Cl. CPC C12Q 1/6886; A61K 31/519; C12Q 2600/156; C12Q 2600/106

(57) ABSTRACT

Biomarkers and methods are disclosed that identify patients being treated with a BTK inhibitor that have acquired a mutation that will cause resistance to the BTK inhibitor. Therefore, also disclosed is a method for treating a hematological cancer in the patient that involves detecting an acquired mutation that causes resistance to a BTK inhibitor and then selecting an alternative treatment if resistance is detected.

### CROSS-REFERENCE TO RELATED APPLICATIONS

This application claims benefit of U.S. Provisional Application No. 62/059,501, filed Oct. 3, 2014, which is hereby incorporated herein by reference in its entirety.

### STATEMENT REGARDING FEDERALLY SPONSORED RESEARCH OR DEVELOPMENT

This invention was made with Government Support under Grant Nos. CA183444, CA177292, and CA140158 awarded by the National Institutes of Health. The Government has certain rights in the invention.

### BACKGROUND

Chronic lymphocytic leukemia (CLL) is the most common leukemia, mostly arising in patients over the age of 50. The disease has been treated with chemo-immunotherapies with varying outcomes, depending on the genetic make-up of the tumor cells. The Bruton tyrosine kinase inhibitor (BTKi) ibrutinib is a new targeted therapy for patients with CLL that induces durable remissions in the majority of CLL patients. However, a small proportion of patients initially responds to the BTKi and then develops resistance. Methods are needed to identify onset of resistance in patients so that alternative treatment options can be selected prior to relapse.

### SUMMARY

Biomarkers and methods are disclosed that identify patients being treated with a BTK inhibitor that have acquired a mutation that will cause resistance to the BTK inhibitor. Therefore, also disclosed is a method for treating a hematological cancer in the patient that involves detecting an acquired mutation that causes resistance to a BTK inhibitor and then selecting an alternative treatment if resistance is detected.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph showing percentage of alleles having the mutations BTK C481 S (), PLCγ2 R665W (▪), PLCγ2 L845F (▴), and PLCγ2 S707Y (▾) as a function of time from start of ibrutinib treatment. Arrow shows time at which ibrutinib treatment ceased.

### DETAILED DESCRIPTION

A method for treating a hematological cancer in a patient is disclosed that involves detection of an acquired mutation that causes resistance to a BTK inhibitor in a blood or tissue sample of the subject and then selecting an alternative treatment if resistance is detected. The method can involve first administering to the patient a composition comprising a therapeutically effective amount of a first Bruton's tyrosine kinase (BTK) inhibitor, e.g., to treat a hematological malignancy, who is at risk of becoming resistant to the BTK inhibitor. Alternatively, the method can involve selecting a patient who is undergoing therapy with a BTK inhibitor and is therefore at risk of becoming resistant to the BTK inhibitor.

B Cell Isolation

The disclosed method involves obtaining a blood or tissue sample from the patient and extracting DNA from the blood or tissue sample for analysis. In some embodiments, B-cells are first enriched from the blood or tissue sample, and the DNA is isolated from enriched B-cells. For example, in some cases, the DNA is extracted from an enriched blood cell population where at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% of the cells are B-cells.

There are multiple methods of B cell isolation known in the art. These include, but are not limited to capture on an antigen-coated solid matrices; rosetting with antigen-coated red blood cells or magnetic particles; and staining with fluorescent antigen and isolation by flow cytometric cell sorting (Kodituwakku et al. Isolation of antigen-specific B cells Immunology and Cell Biology (2003) 81, 163-170; Heine et al. Isolation of Human B Cell Populations. August 2011. Current Protocols in Immunology. John Wiley & Sons, Inc.). The cell population can be enriched to yield purified B cells that are about 50% to about 100% pure, or increments therein. Preferably, the enriched cell population has an antigen-specific cell frequency greater than or equal to about 50%, 60%, 70%, 75%, 80%, 90%, 95%, 96%, 97%, 98%, 99%, or 100% pure.

One method to isolate B cells in bulk is the RosetteSep™ Human B Cell Enrichment Cocktail kit available from StemCell™ Technologies. The RosetteSep™ Human B Cell Enrichment Cocktail is designed to isolate B cells from whole blood by negative selection. Unwanted cells are targeted for removal with Tetrameric Antibody Complexes recognizing CD2, CD3, CD16, CD36, CD56, CD66b and glycophorin A on red blood cells (RBCs). When centrifuged over a buoyant density medium, the unwanted cells pellet along with the RBCs. The purified B cells are present as a highly enriched population at the interface between the plasma and the buoyant density medium.

Another method to isolate B cells is magnetic bead isolation. For example, Dynabeads® Untouched™ Human B Cells Kit (Invitrogen™) can be used. This method isolates pure and viable untouched B cells from PBMC by negative isolation. The kit depletes T cells, NK cells, monocytes, platelets, dendritic cells, granulocytes and erythrocytes. The negatively isolated human B cells are left in the sample and have not been in contact with the Dynabeads®. An antibody mix towards the non-B cells is then added to the sample and allowed to bind to the cells. Dynabeads® are added and will bind to the antibody-labeled cells during a short incubation. The bead-bound cells are quickly separated on a magnet and discarded. The remaining negatively isolated and untouched human B cells can be directly analyzed in a flow cytometer and used for B cell derived DNA isolation.

Another method of isolation of highly purified B cells especially useful in rare cases where blood or bone marrow samples contain both malignant CLL and normal polyclonal B lymphocytes is fluorescence activated cell sorting (FACS). The advantage of FACS sorting is that multiple cell markers can be used simultaneously for positive isolation of cells that express CLL immunophenotype. This technique allows isolation of CLL cells from normal background B lymphocytes and residual T, NK cells and monocytes. These highly purified residual T lymphocytes, NK cells, and monocytes can be used as a good source of germline DNA.

In the rare setting where recovery of normal B-cells occurs greater than 10% of the total recovered B-cells using non-FACS directed isolation, a second application of separation can occur by which malignant B-cells are selected utilizing FACS directed sorting. This further allows enrichment of malignant B-cells to increase selectivity of these to 99+%.

Sequencing

Once isolated, the DNA can be analyzed for onset of BTK resistance by determining partial or complete gene sequences for BTK, PLCγ2, or a combination thereof, to look for an acquired mutation in BTK or PLCγ2 that affects activity of the first BTK inhibitor. This method can be repeated during the course of treatment, e.g., every 1, 2, 3, 4, 5, 6, 7, 8, 9, 10, 11, 12, or more months (including indefinitely), to monitor for the presence of an acquired mutation that affects BTK inhibitor activity and effectiveness. Identification of an acquired mutation in BTK or PLCγ2 that affects BTK inhibitor activity is an indication that the subject is becoming resistant to the BTK inhibitor.

The resistance mutation disclosed herein is an amino acid substitution that can occur in PLCγ2 or BTK, or a gene or transcript mutation encoding the amino acid substitution. In typical embodiments, DNA or RNA extracted from a blood sample is analyzed for the presence of a sequence mutation in PLCγ2 or BTK genes or transcripts that result in protein alterations, such as amino acid substitution, fameshift mutation, truncation, or inversion.

In some embodiments, the presence of a resistance mutation can be conveniently determined using DNA sequencing, including sequencing by synthesis methods, sequencing by ligation, and sequencing by expansion methodologies. Technologies include pyrosequencing, ion semiconductor sequencing, nanopore sequencing, single molecule sequence, e.g. real time single molecule sequencing technology, or other sequencing methods. In some embodiments, single molecule sequencing is employed (e.g., the True Single Molecule Sequencing (tSMS™) sequencing platform (Helicos BioSciences Corporation); or Real Time Single Molecule Sequencing (SMRT™) sequencing platform (Pacific Biosciences Incorporated)).

In some embodiments, the DNA is analyzed by a next-generation sequencing (NGS) method that provides high sensitivity and depth of coverage. In some embodiments, the NGS method has a sensitivity of 1% subclone variant detection. For example, the DNA can be analyzed by ion semiconductor sequencing.

Any of a number hybridization-based assays can also be used to detect a sequence mutation at a codon that encodes one or more of the mutations disclosed herein in nucleic acids obtained from a B cell sample. In some embodiments, DNA or RNA obtained from the B cell sample can be evaluated using known techniques such as allele-specific oligonucleotide hybridization, which relies on distinguishing a mutant position in a nucleic acid from a normal position in a nucleic acid sequence using an oligonucleotide that specifically hybridizes to the mutant or normal nucleic acid sequence. This method typically employs short oligonucleotides, e.g., 15-20 nucleotides, in length, that are designed to differentially hybridize to the normal or mutant allele. Guidance for designing such probes is available in the art. The presence of a mutant allele is determined by measuring the amount of allele-specific oligonucleotide that hybridizes to the sample.

In other embodiments, the presence of a normal or mutant nucleic acid can be detected using allele-specific amplification or primer extension methods. These reactions typically involve use of primers that are designed to specifically target a normal or mutant allele via a mismatch at the 3′ end of a primer. The presence of a mismatch affects the ability of a polymerase to extend a primer when the polymerase lacks error-correcting activity. The amount of amplified product can be determined using a probe or by directly measuring the amount of DNA present in the reaction.

Detection of levels of nucleic acids in a B cell sample that have a mutation at a codon encoding a mutation as disclosed herein can also be performed using a quantitative assay such as a 5′-nuclease activity (also referred to as a “TaqMan@®™” assay), e.g., as described in U.S. Pat. Nos. 5,210,015; 5,487,972; and 5,804,375; and Holland et al., 1988, Proc. Natl. Acad. Sci. USA 88:7276-7280. In such an assay, labeled detection probes that hybridize within the amplified region are added during the amplification reaction. In some embodiments, the hybridization probe can be an allele-specific probe that discriminates a normal or mutant allele. Alternatively, the method can be performed using an allele-specific primer and a labeled probe that binds to amplified product.

Other detection methods include single-stranded conformational analysis, amplicon melting analysis, or methods based on mass spectrometry. Mass spectrometry takes advantage of the unique mass of each of the four nucleotides of DNA. The allele can be unambiguously genotyped by mass spectrometry by measuring the differences in the mass of nucleic acids having alternative PLCγ2 or BTK alleles. MALDI-TOF (Matrix Assisted Laser Desorption Ionization Time of Flight) mass spectrometry technology is preferred for extremely precise determinations of molecular mass, such as single nucleotide mutations. Preferred mass spectrometry-based methods of single nucleotide mutation assays include primer extension assays, which can also be utilized in combination with other approaches, such as traditional gel-based formats and microarrays.

Mutations that Affect BTK Inhibitor Activity

Mutations in BTK and PLCγ2 that affect BTK inhibitor activity are disclosed herein. In some embodiments, the gene mutation encodes an amino acid mutation in BTK selected from the group consisting of a C481S mutation, a C481F mutation, and a P80L mutation. In some embodiments, the mutation is not a C481S mutation in BTK. In some embodiments, the gene mutation encodes an amino acid mutation in PLCγ2 selected from the group consisting of a R665W mutation, a S707Y mutation, a S707P mutation, R742P mutation, L845 frameshift, a L845F mutation, and a D1140G mutation. In some embodiments, the mutation is not a R665W mutation, S707Y mutation, or a L845F mutation in PLCγ2.

Also disclosed are methods for determining whether an acquired mutation affects BTK inhibitor activity and will result in resistance. This method can involve first cloning a cell to stably express a BTK or PLCγ2 gene containing the acquired mutation. This recombinant cell can then be assayed for BTK inhibitor activity, directly or indirectly.

For example, in some embodiments, the method involves contacting the cell with the first BTK inhibitor, and assaying the cell for calcium mobilization, wherein a reduction the ability of the first BTK inhibitor to inhibit calcium flux in the cell compared to a cell not containing the mutation is an indication that the mutation affects activity of the first BTK inhibitor.

In some embodiments, the method involves contacting the cell with the first BTK inhibitor, and assaying the cell for proliferation. Cell proliferation assays are known and are mainly designed based on the following three concepts: (1) measuring rate of DNA replication, (2) analysis of metabolic activity, and (3) cell surface antigen recognitions. Rate of DNA replication can be analyzed by using radioactive or labelled nucleotide analogues, such as ³H-thymidine-based and BrdU-based assays. Metabolic activity-based assays include MTT, XTT, WST, resazurin and ATP measurements. Cell proliferation antigen-based assay targets antigens present in proliferating cells such as markers like Ki-67, topoisomerase IIB, phosphohistone H3, and PCNA.

In some embodiments, the method involves contacting the cell with the first BTK inhibitor, and assaying the cell for surface antigen expression including, but not limited to, CD40, CD80, CD86, CD154, CD69. This assay can also involve BCR stimulation, CpG stimulation, cytokine stimulation, chemokine stimulation, or any combination thereof.

In some embodiments, the method involves contacting the cell with the first BTK inhibitor, and assaying the cell for migration and intracellular signaling changes following BCR stimulation/CpG stimulation/cytokine stimulation/chemokine stimulation.

Selecting BTK Inhibitors

In each of the disclosed embodiments, the method can further involve selecting a second BTK inhibitor for treating the homological cancer if an acquired gene mutation that affects BTK inhibitor activity is detected. For example, the first BTK inhibitor can be Ibrutinib, and the second BTK inhibitor can be a drug that does not bind BTK at amino acid residue C481.

Ibrutinib (PCI-32765; 1-[(3R)-3-[4-amino-3-(4-phenoxyphenyl)pyrazolo[3,4-d]pyrimidin-1-yl]piper-idin-1-yl]prop-2-en-1-one)) is an orally bioavailable, first-in-class, highly potent small molecule inhibitor with subnanomolar activity (IC₅₀, 0.5 nM) against BTK. It selectively binds to Cys-481 residue in the allosteric inhibitory segment of BTK (TK/SH1 domain), and irreversibly blocks its enzymatic activity. The compound also abrogates the full activation of BTK by inhibiting its autophosphorylation at Tyr-223.

Other BTK inhibitors include CC-292, ONO-4059, ACP-196, RN486, HM-71224, CGI-1746, GDC-0834, CGI-560, CNX-774, and LFM-A13. BTK inhibitors that bind C481, such as CC-292, ONO-4059, and ACP-196, would in some embodiments not be used as second BTK inhibitors to treat Ibrutinib resistant patients unless pharmacology as a reversible inhibitor could assure adequate coverage of target inhibition. However, non-C481-binding BTK inhibitors, such as RN486, HM-71224, and CGI-1746, could be used to treat Ibrutinib resistant patients.

CC-292 (N-(3-((5-fluoro-2-((4-(2-methoxyethoxy)phenyl)amino)pyrimidin-4-yl)amino)phenyl)acrylamide) is an orally bioavailable acrylamide derivative with potent, irreversible anti-BTK activity (IC₅₀<0.5 nM) in biochemical kinase assays. The small molecule inhibitor abolishes BCR signaling in Ramos human Burkitt's lymphoma cell line by covalently binding to BTK, and selectively inhibits its autophosphorylation as well as activation of PLCγ2 and other downstream substrates of BTK.

ONO-4059 is a highly selective, orally bioavailable inhibitor of BTK kinase activity with a potency (IC₅₀) of 2.2 nM. The compound covalently binds to BTK, and reversibly blocks BCR signaling and B-cell proliferation and activation.

ACP-196 is an orally available BTK inhibitor.

RN-486 (6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one) demonstrates subnanomolar and highly specific activity against purified BTK in enzymatic assays.

HM-71224 is an oral, small molecule BTK inhibitor that is being developed by Hanmi pharmaceuticals.

CGI-1746 (N-[3-[4,5-Dihydro-4-methyl-6-[[4-(4-morpholinylcarbonyl)phenyl]amino]-5-oxo-2-pyrazinyl]-2-methylphenyl]-4-(tert-butyl)benzamide) is a selective and ATP-competitive small molecule inhibitor with unique BTK-inhibitory property that potently inhibits both auto- and trans-phosphorylation of BTK. It binds and occupies an SH3 binding pocket within the un-phosphorylated BTK and stabilizes it in this inactive enzyme conformation state.

GDC-0834 ((R)-N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide) is a potent, highly selective, reversible BTK inhibitor with nanomolar activity in enzyme kinetics studies.

CGI-560 (4-(tert-butyl)-N-(3-(8-(phenylamino)imidazo[1,2-a]pyrazin-6-yl)phenyl)benzamide), a benzamide derivative, is a highly selective (>10 fold) but modestly potent small molecule inhibitor of BTK with an IC₅₀ of 400 nM in enzymology assays. Optimization of CGI-560 property by medicinal chemistry led to the discovery of another benzamide analogue (CGI-1746) with exquisite potency and unique BTK-inhibitory activity.

CNX-774 (4-(4-((4-((3-acrylamidophenyl)amino)-5-fluoropyrimidin-2-yl)amino)phenoxy)-N-methylpicolinamide) is another orally available, small molecule inhibitor with irreversible BTK-inhibitory property. CNX-774 is highly selective for BTK, and forms a ligand-directed covalent bond with the Cys-481 residue within the ATP binding site of the enzyme. In biochemical and cellular assays, CNX-774 demonstrates potent inhibitory activity towards BTK with an IC₅₀ of <1 nM and 1-10 nM respectively.

LFM-A13 (2-Cyano-N-(2,5-dibromophenyl)-3-hydroxy-2-butenamide) is a novel, first-in-class, dual BTK/Polo-like kinases (PLK) inhibitor with anti-proliferative, pro-apoptotic, and chemosensitising effects in leukemia/lymphoma and breast cancer cells. A leflunomide metabolite analogue, LFM-A13 binds favorably to the catalytic site within the kinase domain of BTK, and exhibits an inhibitory potency (IC₅₀) of 17.2 microM in cell-free kinase assays.

Additional BTK inhibitor agents include AVL-291; AVL-292; PCI-45292; 6-phenyl-imidazo[1,2-a]pyridine; 6-phenyl-imidazo[1,2-b]pyridazine derivatives as described in U.S. Pat. No. 8,324,211; pyridinone and pyridazinone derivatives as described in U.S. Pat. No. 8,318,719; 3-amino-5-phenyl-1H-pyridin-2-one derivatives as described in U.S. Pat. No. 8,299,077; 1H-pyrazolo(3,4-d)pyrimidin-4-ylamine substitutes as described in U.S. Pat. No. 8,232,280; 3-phenyl-1H-pyrazolo-pyrimidin-4-ylamine substitutes as described in U.S. Pat. No. 8,236,812; compounds as described in U.S. Pat. No. 7,393,848; U.S. Pat. No. 7,405,295; U.S. Pat. No. 7,514,444; U.S. Pat. No. 7,625,880; U.S. Pat. No. 7,683,064; U.S. Pat. No. 7,732,454; U.S. Pat. No. 7,741,330; U.S. Pat. No. 7,825,118; U.S. Pat. No. 7,902,194; U.S. Pat. No. 7,906,509; U.S. Pat. No. 7,943,618; U.S. Pat. No. 7,960,396; U.S. Pat. No. 8,008,309; U.S. Pat. No. 8,067,395; U.S. Pat. No. 8,088,781; U.S. Pat. No. 8,124,604; US20130018032; US20130018060, and US20130035334, the disclosures of which are incorporated herein by reference thereto.

Likewise, inhibitors that demonstrate the ability to lock BTK in its inactive state such as the tool molecule suggested in Nat Chem Biol. 7:41-50, 2011 could be used to treat Ibrutinib resistant patients. Additionally, agents that promote degradation of mutant BTK proteins such as HSP90 inhibitors would represent a strategy to add to patients having these BTK mutations. For PLCG2 mutations, consideration of targeting down-stream targets such as PCK-B would be considered. Additionally, one can target pathways that are independent of BTK and PLCG2. In some embodiments, the patient can be treated with a cyclin dependent kinase inhibitor, PI3-kinase inhibitor, XPO1 inhibitor, ABT-199, or a combination thereof. In addition, therapeutic antibodies such as Obinutuzumab, MOR028, Rituximab, Alemtuzumab, Ofatumumab, CD37 antibodies, and BAF-R ab can be used.

B-Cell Related Diseases

Disclosed herein, in certain embodiments, is a method for treating a hematological malignancy in an individual in need thereof. In some embodiments, the hematological malignancy is a chronic lymphocytic leukemia (CLL), small lymphocytic lymphoma (SLL), high risk CLL, or a non-CLL/SLL lymphoma. In some embodiments, the hematological malignancy is follicular lymphoma, diffuse large B-cell lymphoma (DLBCL), mantle cell lymphoma, Waldenstrom's macroglobulinemia, multiple myeloma, marginal zone lymphoma, Burkitt's lymphoma, non-Burkitt high grade B cell lymphoma, hairy cell leukemia, prolymphocytic leukemia, or extranodal marginal zone B cell lymphoma. In some embodiments, the hematological malignancy is acute or chronic myelogenous (or myeloid) leukemia, myelodysplastic syndrome, or acute lymphoblastic leukemia. In some embodiments, the hematological malignancy is relapsed or refractory diffuse large B-cell lymphoma (DLBCL), relapsed or refractory mantle cell lymphoma, relapsed or refractory follicular lymphoma, relapsed or refractory CLL; relapsed or refractory SLL; relapsed or refractory multiple myeloma. In some embodiments, the hematological malignancy is a hematological malignancy that is classified as high-risk. In some embodiments, the hematological malignancy is high risk CLL or high risk SLL.

B-cell lymphoproliferative disorders (BCLDs) are neoplasms of the blood and encompass, inter alia, non-Hodgkin lymphoma, multiple myeloma, and leukemia. BCLDs can originate either in the lymphatic tissues (as in the case of lymphoma) or in the bone marrow (as in the case of leukemia and myeloma), and they all are involved with the uncontrolled growth of lymphocytes or white blood cells. There are many subtypes of BCLD, e.g., chronic lymphocytic leukemia (CLL) and non-Hodgkin lymphoma (NHL).

Disclosed herein, in certain embodiments, is a method for treating a non-Hodgkin's lymphoma in an individual in need thereof. Further disclosed herein, in certain embodiments, is a method for treating relapsed or refractory non-Hodgkin's lymphoma in an individual in need thereof. In some embodiments, the non-Hodgkin's lymphoma is relapsed or refractory diffuse large B-cell lymphoma (DLBCL), relapsed or refractory mantle cell lymphoma, or relapsed or refractory follicular lymphoma.

A non-limiting list of the B-cell NHL includes Burkitt's lymphoma (e.g., Endemic Burkitt's Lymphoma and Sporadic Burkitt's Lymphoma), Cutaneous B-Cell Lymphoma, Cutaneous Marginal Zone Lymphoma (MZL), Diffuse Large Cell Lymphoma (DLBCL), Diffuse Mixed Small and Large Cell Lymphoma, Diffuse Small Cleaved Cell, Diffuse Small Lymphocytic Lymphoma, Extranodal Marginal Zone B-cell lymphoma, follicular lymphoma, Follicular Small Cleaved Cell (Grade 1), Follicular Mixed Small Cleaved and Large Cell (Grade 2), Follicular Large Cell (Grade 3), Intravascular Large B-Cell Lymphoma, Intravascular Lymphomatosis, Large Cell Immunoblastic Lymphoma, Large Cell Lymphoma (LCL), Lymphoblastic Lymphoma, MALT Lymphoma, Mantle Cell Lymphoma (MCL), immunoblastic large cell lymphoma, precursor B-lymphoblastic lymphoma, mantle cell lymphoma, chronic lymphocytic leukemia (CLL)/small lymphocytic lymphoma (SLL), extranodal marginal zone B-cell lymphoma-mucosa-associated lymphoid tissue (MALT) lymphoma, Mediastinal Large B-Cell Lymphoma, nodal marginal zone B-cell lymphoma, splenic marginal zone B-cell lymphoma, primary mediastinal B-cell lymphoma, lymphoplasmocytic lymphoma, hairy cell leukemia, Waldenstrom's Macroglobulinemia, and primary central nervous system (CNS) lymphoma. Additional non-Hodgkin's lymphomas are contemplated within the scope of the present invention and apparent to those of ordinary skill in the art.

Disclosed herein, in certain embodiments, is a method for treating a Diffuse large B-cell lymphoma (DLCBL) in an individual in need thereof. As used herein, the term “Diffuse large B-cell lymphoma (DLBCL)” refers to a neoplasm of the germinal center B lymphocytes with a diffuse growth pattern and a high-intermediate proliferation index. Disclosed herein, in certain embodiments, is a method for treating diffuse large B-cell lymphoma, activated B cell-like subtype (ABC-DLBCL), in an individual in need thereof.

Disclosed herein, in certain embodiments, is a method for treating a follicular lymphoma in an individual in need thereof. As used herein, the term “follicular lymphoma” refers to any of several types of non-Hodgkin's lymphoma in which the lymphomatous cells are clustered into nodules or follicles.

Disclosed herein, in certain embodiments, is a method for treating Chronic lymphocytic leukemia or small lymphocytic lymphoma (CLL/SLL) in an individual in need thereof. Chronic lymphocytic leukemia and small lymphocytic lymphoma (CLL/SLL) are commonly thought as the same disease with slightly different manifestations. Where the cancerous cells gather determines whether it is called CLL or SLL. When the cancer cells are primarily found in the lymph nodes, lima bean shaped structures of the lymphatic system (a system primarily of tiny vessels found in the body), it is called SLL.

Disclosed herein, in certain embodiments, is a method for treating a Mantle cell lymphoma in an individual in need thereof. As used herein, the term, “Mantle cell lymphoma” refers to a subtype of B-cell lymphoma, due to CD5 positive antigen-naive pregerminal center B-cell within the mantle zone that surrounds normal germinal center follicles. MCL cells generally over-express cyclin D1 due to a t(11:14) chromosomal translocation in the DNA. More specifically, the translocation is at t(11;14)(q13;q32).

Disclosed herein, in certain embodiments, is a method for treating a marginal zone B-cell lymphoma in an individual in need thereof. As used herein, the term “marginal zone B-cell lymphoma” refers to a group of related B-cell neoplasms that involve the lymphoid tissues in the marginal zone, the patchy area outside the follicular mantle zone. Marginal zone lymphomas account for about 5% to 10% of lymphomas. The cells in these lymphomas look small under the microscope. There are 3 main types of marginal zone lymphomas including extranodal marginal zone B-cell lymphomas, nodal marginal zone B-cell lymphoma, and splenic marginal zone lymphoma.

Disclosed herein, in certain embodiments, is a method for treating a MALT in an individual in need thereof. The term “mucosa-associated lymphoid tissue (MALT) lymphoma”, as used herein, refers to extranodal manifestations of marginal-zone lymphomas.

Disclosed herein, in certain embodiments, is a method for treating a nodal marginal zone B-cell lymphoma in an individual in need thereof. The term “nodal marginal zone B-cell lymphoma” refers to an indolent B-cell lymphoma that is found mostly in the lymph nodes.

Disclosed herein, in certain embodiments, is a method for treating a splenic marginal zone B-cell lymphoma. The term “splenic marginal zone B-cell lymphoma” refers to specific low-grade small B-cell lymphoma that is incorporated in the World Health Organization classification.

Disclosed herein, in certain embodiments, is a method for treating a Burkitt lymphoma in an individual in need thereof. The term “Burkitt lymphoma” refers to a type of Non-Hodgkin Lymphoma (NHL) that commonly affects children. There are two classifications, endemic Burkitt's lymphoma and sporadic Burkitt's lymphoma:

Disclosed herein, in certain embodiments, is a method for treating a Waldenstrom macroglobulinemia. The term “Waldenstrom macroglobulinemia”, also known as lymphoplasmacytic lymphoma, is cancer involving a subtype of white blood cells called lymphocytes.

Disclosed herein, in certain embodiments, is a method for treating multiple myeloma in an individual in need thereof. Multiple myeloma, also known as MM, myeloma, plasma cell myeloma, or as Kahler's disease (after Otto Kahler) is a cancer of the white blood cells known as plasma cells.

Disclosed herein, in certain embodiments, is a method for treating a leukemia in an individual in need thereof. Leukemia is a cancer of the blood or bone marrow characterized by an abnormal increase of blood cells, usually leukocytes (white blood cells). Leukemia is a broad term covering a spectrum of diseases. The first division is between its acute and chronic forms: (i) acute leukemia is characterized by the rapid increase of immature blood cells. This crowding makes the bone marrow unable to produce healthy blood cells. Immediate treatment is required in acute leukemia due to the rapid progression and accumulation of the malignant cells, which then spill over into the bloodstream and spread to other organs of the body. Acute forms of leukemia are the most common forms of leukemia in children; (ii) chronic leukemia is distinguished by the excessive buildup of relatively mature, but still abnormal, white blood cells. Typically taking months or years to progress, the cells are produced at a much higher rate than normal cells, resulting in many abnormal white blood cells in the blood. Chronic leukemia mostly occurs in older people, but can theoretically occur in any age group. Additionally, the diseases are subdivided according to which kind of blood cell is affected. This split divides leukemias into lymphoblastic or lymphocytic leukemias and myeloid or myelogenous leukemias: (i) lymphoblastic or lymphocytic leukemias, the cancerous change takes place in a type of marrow cell that normally goes on to form lymphocytes, which are infection-fighting immune system cells; (ii) myeloid or myelogenous leukemias, the cancerous change takes place in a type of marrow cell that normally goes on to form red blood cells, some other types of white cells, and platelets.

Within these main categories, there are several subcategories including, but not limited to, Acute lymphoblastic leukemia (ALL), Acute myelogenous leukemia (AML), Chronic myelogenous leukemia (CML), and Hairy cell leukemia (HCL).

Symptoms, diagnostic tests, and prognostic tests for each of the above-mentioned conditions are known in the art. See, e.g., Harrison's Principles of Internal Medicine®,” 16th ed., 2004, The McGraw-Hill Companies, Inc. Dey et al. (2006), Cytojournal 3(24), and the “Revised European American Lymphoma” (REAL) classification system (see, e.g., the website maintained by the National Cancer Institute).

### Definitions

The term “B cell” refers to a lymphocyte that matures within the bone marrow, and includes a naive B cell, memory B cell, or effector B cell (plasma cell). The B cell herein is a normal or non-malignant B cell.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder, and supportive treatment, that is, treatment employed to supplement another specific therapy directed toward the improvement of the associated disease, pathological condition, or disorder.

Therefore, a patient in need of treatment includes a patient already with a B-cell related disease, as well as those in which the B-cell related disease or the progress of the B-cell related disease is to be prevented. Hence, the subject may have been diagnosed as having the B-cell related disease, or may be predisposed or susceptible to the B-cell related disease, or may have B-cell related disease that is likely to progress in the absence of treatment. Treatment is successful herein if the B-cell related disease is alleviated or healed, or progression of B-cell related disease, including its signs and symptoms, is halted or slowed down as compared to the condition of the subject prior to administration. Successful treatment further includes complete or partial prevention of the B-cell related disease. For purposes herein, slowing down or reducing B-cell related disease or the progression of the B-cell related disease is the same as arrest, decrease, or reversal of the B-cell related disease. The effectiveness of treatment of the B-cell related disease in the method can, for example, be determined by using clinical response parameters in the subjects with B-cell related disease, or by assaying a molecular determinant of the degree of the B-cell related disease in the subject. A clinician may use any of several methods known in the art to measure the effectiveness of a particular dosage scheme. Dosage regimens may be adjusted to provide the optimum desired response (e.g., a therapeutic response). For example, a dose may be administered, several divided doses may be administered over time, or the dose may be proportionally reduced or increased as indicated by exigencies of the therapeutic situation.

An “effective response” of a subject or a subject's “responsiveness” to treatment described herein and similar wording refers to the clinical or therapeutic benefit imparted to a subject not responsive to the treatment from or as a result of the treatment. Such benefit includes cellular or biological responses, a complete response, a partial response, a stable disease (without progression or relapse), or a response with a later relapse of the subject from or as a result of the treatment.

The term “further treated”, “further administer” or “further administered”, means that the different therapeutic agents may be administered subsequently, intermittently or one after the other. Such further administration may be temporally separated, for example at different times, on different days, and via different modes or routes of administration.

The term “clinical improvement” refers to prevention of further progress of the B-cell related disease or any improvement in the B-cell related disease as a result of treatment, as determined by various testing.

For purposes herein, a subject is in “remission” if he/she has no symptoms of the B-cell related disease, such as malignancy and has had no progression of the B-cell related disease, such as malignancy, as assessed at baseline or at a certain point of time during treatment. Those who are not in remission include, for example, those experiencing a worsening or progression of the B-cell related disease. Such subjects experiencing a return of symptoms, including active B-cell malignancy, are those who are “non-responsive” or have “relapsed” or had a “recurrence.”

In the present application, “isolating” or “enriching” a cell population cells means increasing the frequency of desired cells contained in a mixed cell population, e.g., a B cell-containing isolate derived from a host. Thus, an isolated cell population encompasses a cell population having a higher frequency of B cells, for example as a result of an enrichment or isolation step.

The general term “cell population” encompasses pre- and a post-enrichment cell populations, keeping in mind that when multiple isolation steps are performed, a cell population can be both pre- and post-enrichment.

The term “depth of coverage” refers to the average number of times a single base is sequenced during the sequencing run. The term also describes how much of the complexity in a sequencing library has been sampled. A DNA sample contains finite pools of distinct DNA fragments. In a sequencing experiment only some of these fragments are sampled. The number of these distinct fragments sequenced is positively correlated with the depth of the true biological variation that has been sampled.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: High Sensitivity and Specificity Human BTK and PLCG2 Genes Mutation Detection Test Using Ion Torrent Next Generation Sequencing Platform

Mutations of BTK and PLCG2 genes were initially detected using a comprehensive chronic lymphocytic leukemia (CLL) mutation screening panel. Based on these initial findings short Ion Torrent panel focused on BTK and PLCγ2 genes was designed. Performance of BTK and PLCγ2 focused panel was evaluated and validated by analysis of cases with known BTK and PLCγ2 mutations. Focused panel showed 100% concordance when compared with comprehensive CLL panel results as well as with Sanger sequencing results. Moreover for samples with % of mutation lower that that detectable by Sanger focused panel show high sensitivity and specificity as indicated by subsequent samples with higher % of mutation. Focused BTK and PLCγ2 panel show no evidence of mutations for all analyzed samples that were negative for BTK and PLCγ2 mutations. Based on these findings focused BTK and PLCγ2 mutation detection panel is sensitive (e.g., 3% of variant detection by algorithm and 2% by manual review) and specific (e.g., no false negative or false positive results).

Table 1 is a list of mutations identified in ibrutinib-resistant patients using Ion Torrent to sequence DNA from peripheral blood cells.

TABLE 1
Ibrutinib-resistance mutations

| | | | | Baseline | | Relapse | |
|---|---|---|---|---|---|---|---|
| | | | | | Variant | | Variant |
| Patient | Chromosome | Gene | mutation | Coverage | Frequency | Coverage | Frequency |
| 1 | 16 | PLCγ2 | R665W | 870 | 0 | 774 | 3.6 |
| | 16 | PLCγ2 | S707P | 2940 | 0 | 1994 | 5.3 |
| | 16 | PLCγ2 | S707F | 2937 | 0 | 1999 | 4.4 |
| | 16 | PLCγ2 | R742P | 1623 | 0 | 1630 | 5.5 |
| | 16 | PLCγ2 | L845fs | 1200 | 0 | 1394 | 14.8 |
| 2 | 16 | PLCγ2 | R665W | 955 | 0 | 500 | 44.4 |
| 3 | X | BTK | C481F | 1837 | 0 | 1987 | 80.5 |
| 4 | X | BTK | C481S | 693 | 0 | 978 | 13.5 |
| 5 | 16 | PLCγ2 | R665W | 928 | 0 | 614 | 3.7 |
| | 16 | PLCγ2 | S707Y | 2839 | 0 | 1287 | 6.8 |
| | 16 | PLCγ2 | L845F | 207 | 0 | 806 | 23.8 |
| | X | BTK | C481S | 875 | 0 | 1011 | 3.5 |
| 6 | X | BTK | C481S | 2086 | 0 | 440 | 44.3 |
| 7 | X | BTK | C481S | 305 | 2 | 490 | 47.5 |
| 8 | X | BTK | C481S | 1210 | 0 | 1690 | 72 |
| 9 | X | BTK | C481S | 2260 | 0 | 1830 | 2 |
| 10 | 16 | PLCγ2 | D1140G | 1372 | 0 | 1288 | 5.1 |
| | X | BTK | C481S | 3109 | 0 | 1999 | 4.9 |
| 11 | X | BTK | C481S | 1119 | 0 | 930 | 4.2/4.1 |

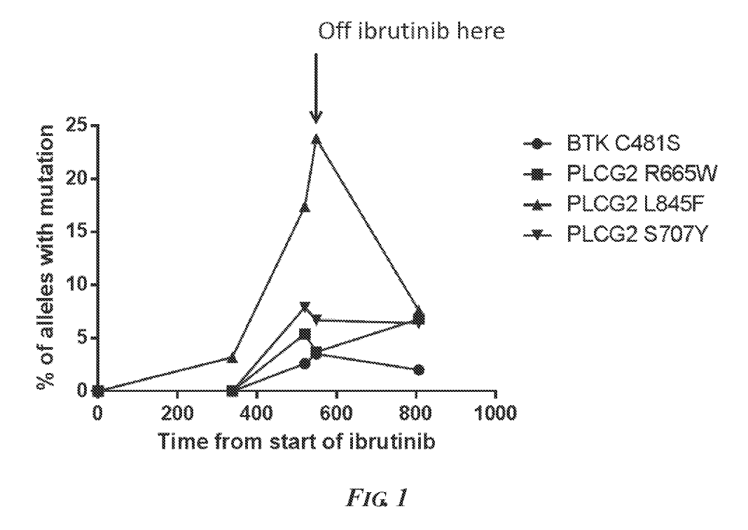

FIG. 1 is a graph showing percentage of alleles having the mutations BTK C481S (). PLCγ2 R665W (▪), PLCγ2 L845F (▴), and PLCγ2 S707Y (▾) as a function of time from start of ibrutinib treatment. Arrow shows time at which ibrutinib treatment ceased.

Heterozygous mutations in BTK C481S and PLCγ2 D1140G were identified in DNA isolated from enriched B-cells (by CD19 expression) of patients with ibrutinib-resistance.

Table 2A and 2B show examples of mutations detected in ibrutinib-resistant patients using DNA isolated from enriched B-cells (by CD19 expression).

TABLE 2A
Ibrutinib-resistance mutations in patient 934 using DNA from enriched B-cells

| | | | | | | | | | | | | | Original |
|---|---|---|---|---|---|---|---|---|---|---|---|---|---|
| | | | | Var- | | | Fre- | | | Allele | Gene | | Cover- |
| Chrom | Position | Exon | Ref | iant | mutation | Allele Call | quency | Quality | Type | Source | ID | Region Name | age |
| chr16 | 81973602 | 30 | A | G | D1140G | Heterozygous | 13.5 | 3268.17 | SNP | Novel | PLCG2 | AMPL7153068581 | 5966 |
| chrX | 100611164 | 15 | C | G | C481S | Heterozygous | 13 | 3533.56 | SNP | Novel | BTK | AMPL7153305190 | 6942 |

TABLE 2B
Ibrutinib-resistance mutations in patient 164 using DNA from enriched B-cells

| | | | | Var- | AA | | | | | Gene | | Original | Cover- |
|---|---|---|---|---|---|---|---|---|---|---|---|---|---|
| Chrom | Position | Exon | Ref | iant | change | Allele Call | Frequency | Quality | Type | ID | Region Name | Coverage | age |
| chrX | 100611164 | 15 | C | G | C481S | Heterozygous | 5.4 | 556.41 | SNP | BTK | AMPL7153305190 | 9318 | 9308 |
| chrX | 100611165 | 15 | A | T | C481S | Heterozygous | 3.4 | 69.93 | SNP | BTK | AMPL7153305190 | 9270 | 9275 |

Materials and Methods

Ion Torrent Analysis

DNA was extracted from cryopreserved isolated CLL cells using DNA extraction kit (QIAamp DNA Mini Kit from Qiagen) according to manufacturer recommendations. DNA was quantified using spectrophotometric method (Nanodrop 2000 from Thermo Scientific) using standard 260/280 OD ratio. Analysis of the BTK and PLC-gamma genes was performed using next generation sequencing Ion Torrent platform and reagents from Life Technologies (Carlsbad, Calif.). Library was prepared with Ion AmpliSeq Library kit2.0 (4475345) with custom designed panel of AmpliSeq primers (panel design IAD48992, pipe line version 3.0, 87 amplicons in 2 pools, 17 kb panel size, 99.68% coverage) that covers the entire coding sequence and intronic splice acceptor and donor sites for both genes and IonExpress barcode adapters (kit#4471250 and #4474009). DNA was amplified on GeneAmp PCR system 9700 Dual 96-well thermal cycler from Applied Biosystems. PCR product was purified with Agencourt AMPure XP kit (A63881 Beckman Coulter, Indianapolis, Ind.). Library was quantified using real time PCR with Ion Library TAQMAN Quantitation kit 44688022 on (Applied Biosystems ViiA7 Real Time PCR System) instrument to allow for optimal final dilution of library for template preparation on OneTouch OT2 version instrument with Ion PGM Template OT2 200Kit (4480974). The ISPs enrichment and purification was performed on Ion One Touch2 ES.Purified ISPs were analyzed on Ion Torrent personal Genome Machine using IonPGM Sequencing 200v2 kit (4482006) and 318 chips v2 (4484354). Data was collected and analyzed using Torrent Server (4462616) with Torrent Suite 4.0.2 version. Final analysis of sequence data was performed using combination of software: Variant Caller v.4.0-r76860, IGV3.6.033 and IonReporter v.4.0. The following reference sequences were used for analysis; for BTK NM000061.2 and for PLCG2 NM002661.3. The entire length of sequences was reviewed manually using these programs to asses for deviation from reference sequence and to evaluate the quality of sequence and the depth of coverage. The depth of coverage ranged from 1000 to 15000 for different amplicons.
PGM Serial #11C121203
Ion OneTouch OT2 Serial#2456276-0901
Ion OneTouch 2 ES Serial#396398
AmpliSeq BTK/PLCG2 panel design IAD 48992 received Dec. 10, 2013
Human genome version: Hg19
Pipe line version: 3.0
Exon padding 50 bp
In 2 pools
87 amplicons
174 primers
17 kb panel size
Coverage Total: 99.68%
   BTK: 99.44%
   PLCG2: 99.81%
Library Preparation:
15 ng of genomic DNA
Ion AmpliSeq Library Kit2.0 (Life technologies #4475345)
Ion Express Barcode kit (Life Technologies #4471250 and #4474009)
PCR:
   PCR using GeneAmp PCR System 9700 Dual 96-well Thermal Cycler
      Hold, 99° C.-2 min
      21 cycles @99° C. for 15 sec
      60° C. for 4 min
      Hold, 10° C. inf.
Cleaning: with Agencourt AMPure XPkit
Amplification: ×3
Real time PCR using Ion Library Quantitation Kit #4468802
Applied Biosystems ViiA7 Real Time PCR System
Template preparation using Ion OneTouch OT2 with 10 pM library input
Ion PGM Template OT2 200Kit (Life Technologies #4480974)
Enrichment using Ion OneTouch 2 ES
Sequencing:
Ion PGM sequencing 200kit v.2 (Life Technologies item no. 4482006)
Ion 318 chip kit v2 (Life Technologies item no. 4484354) with loading 8 samples per chip
Resulting in medium depths of coverage 6,038
Run uniformity 95%

Amplicon coverage: ranging from 1000-15,000 with 8 barcoded samples run on 318 chip. The average depth of coverage can be increased if desired by reducing the number of samples run on 318 chip with attainable depth of coverage of hot spots of 25,000 for 2 barcoded samples run on 318 chip.
For BTK hotspot
(@100609666 (L528) average 2251 reads
(100611164 (C481) average 5074 reads
For PLCG hotspots
@81946260 (R655) average 5418 reads
(81953154 (S707) average 6503 reads
(@81962183 (L845) average 3172 reads
Analysis: Raw data using Ion Torrent Suite Version 4.0.2
Reanalyzed by Variant Caller Version 4.0-r76860
Settings: Somatic, low stringency
Reanalysis using Ion reporter Version 4.0
Cell preparation and DNA isolation:

To enhance mutation detection sensitivity and to prevent CLL cells DNA dilution by non malignant residual leukocytes including T lymphocytes, NK cells, monocytes and myeloid cells this test is intended to use with purified B lymphocytes. This step is important especially for blood samples with low number of circulating CLL cells where DNA contribution of nonmalignant cells may be substantial. CLL cells can be isolated using any of commercially available methods for positive B cell isolation from whole blood sample anticoagulated with EDTA (purple top tube) or ACDA (yellow top tube). Heparinized blood should be avoided due to potential interference of heparin with multiplex PCR during library preparation. It is important to choose B cell isolation method that is compatible with subsequent DNA isolation methodology used in laboratory. To assure maximum sensitivity of this test B cell isolates should have minimum purity of 80% B cells. The absolute number of isolated B cells should be sufficient to produce 50 ng of genomic DNA. Focused BTK/PLCG test requires 15 ng of DNA, however, 50 ng of total DNA is recommended to assure sufficient amount of DNA for potential repeats of BTK/PLCG2 Ion Torrent testing and if necessary for subsequent Sanger sequencing analysis confirmation of detected variants. In general 50 ng of DNA can be isolated from 50,000 cells using different DNA isolation commercial kits.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for treating a hematological cancer in a subject, comprising:
(a) administering to the subject a composition comprising a therapeutically effective amount of a first Bruton's tyrosine kinase (BTK) inhibitor;
(b) obtaining a blood sample from the subject, isolating B-cells from the blood sample, and extracting DNA from the B-cells;
(c) analyzing the DNA to identify a partial or complete gene sequences for BTK, PLCγ2, or a combination thereof; and
(d) repeating steps (b) and (c) to monitor for the presence of an acquired mutation in BTK or PLCγ2 that affects activity of the first BTK inhibitor;
wherein the presence of acquired mutation in BTK or PLCγ2 that affects BTK inhibitor activity is an indication that the subject is becoming resistant to the BTK inhibitor.

2. The method of claim 1, wherein the acquired mutation encodes an amino acid mutation in BTK selected from the group consisting of a C481S mutation, a C481F mutation, and a P80L mutation.

3. The method of claim 1, wherein the acquired mutation encodes an amino acid mutation in PLCγ2 selected from the group consisting of a R665W mutation, a S707Y mutation, a S707P mutation, R742P mutation, L845 frameshift, a L845F mutation, and a D1140G mutation.

4. The method of claim 1, wherein the DNA is extracted from a blood or tissue cell population where at least 80% of the cells are B-cells.

5. A method for treating a hematological cancer in a subject, comprising
(a) administering to the subject a composition comprising a therapeutically effective amount of a first Bruton's tyrosine kinase (BTK) inhibitor;
(b) obtaining a blood or tissue sample from the subject and extracting DNA therefrom;
(c) analyzing the DNA to identify a partial or complete gene sequences for BTK, PLCγ2, or a combination thereof; and
(d) repeating steps (b) and (c) to monitor for the presence of an acquired mutation in BTK or PLCγ2 that affects activity of the first BTK inhibitor,
wherein the acquired gene mutation encodes an amino acid mutation other than a C481S mutation in BTK, a R665W mutation in PLCγ2, a S707Y mutation in PLC-2, or a L845F mutation in PLCγ2;
wherein the presence of an acquired mutation in BTK or PLCγ2 that affects BTK inhibitor activity is an indication that the subject is becoming resistant to the BTK inhibitor.

6. The method of claim 1, further comprising selecting a second BTK inhibitor for treating the homological cancer if an acquired gene mutation that affects BTK inhibitor activity is detected.

7. The method of claim 1, wherein the first BTK inhibitor comprises Ibrutinib, wherein the second BTK inhibitor comprises a drug that does not bind BTK at amino acid residue C481.

8. The method of claim 1, wherein the DNA is analyzed by ion semiconductor sequencing.

9. The method of claim 1, wherein the hematological cancer comprises a B-cell leukemia or lymphoma.

10. The method of claim 9, wherein the hematological cancer comprises a chronic lymphocytic leukemia (CLL).

11. The method of claim 9, wherein the hematological cancer is selected from the group consisting of mantle cell lymphoma (MCL), acute myeloid leukemia (AML), acute lymphoblastic leukemia (ALL), Waldenstrom's macroglobulinemia, diffuse large B cell lymphoma, follicular lymphoma, marginal zone lymphoma, hairy cell leukemia, and prolymphocytic leukemia.

12. The method of claim 1, wherein the acquired mutation encodes an amino acid mutation in BTK selected from the group consisting of a C481F mutation and a P80L mutation.

13. The method of claim 1, wherein the acquired mutation encodes an amino acid mutation in PLCγ2 selected from the group consisting of a S707P mutation, R742P mutation, L845 frameshift, and a D1140G mutation.
